(19) United States
(12) Patent Application Publication
(10) Pub. No.: US 2022/0265319 A1
(43) Pub. Date: Aug. 25, 2022

(54) PROTECTION DEVICE FOR MEDICAL DEVICE

(71) Applicant: ANECOVA S.A., Lausanne (CH)

(72) Inventors: Mathieu ASTRANSKAS, Etoy (CH); Peter Christian SVALANDER, Etoy (CH); Alison VELASCO, Prilly (CH); Martin VELASCO, Perroy (CH)

(21) Appl. No.: 17/596,982

(22) PCT Filed: Jun. 22, 2020
(86) PCT No.: PCT/EP2020/067269

§ 371 Date: Dec. 22, 2021

(30) Foreign Application Priority Data
Jun. 24, 2019 (FR) FR1906812

(51) Int. Cl. A61B 17/435; A61B 50/30

(52) U.S. Cl. CPC A61B 17/435; A61B 50/30; A61B 2050/0016

(57) ABSTRACT

Disclosed is a protection device for a medical device comprising a substantially cylindrical body and a tube, the body and tube being hollow and open at their ends, the body comprising a thermally insulating distal part and assembly means, the tube comprising complementary assembly means, the body being capable of sliding on the tube in a longitudinal direction from a first position corresponding to a sampling configuration of the protection device to a second position corresponding to a protection configuration of the protection device, the assembly means of the body and the complementary assembly means being capable of immobilising the body in longitudinal translation in the first position and the second position.

The present invention relates to a protection device for a medical device.

It also relates to a medical transfer assembly comprising the protection device and a transfer device as a medical device.

Finally, it relates to a method for the transfer of an element to be sampled by means of the medical transfer assembly.

During certain medical interventions, practitioners are involved in handling medical devices. These may be for example, devices for the transfer of biological material, such as an embryo.

In this specific context, biological material is sampled in the laboratory by means of the transfer device from a preparation arranged for example in medium in a Petri dish. Sampling is generally carried out in a temperature-controlled environment, for example a temperature close to that of the human body, i.e. approximately 37° C. Sampling is carried out for example in a transparent heated cabinet. The cabinet can comprise two holes suitable for allowing the arms of a practitioner to pass through, so as to permit sampling of the biological material within the cabinet. The transfer device is thus maintained at a predefined controlled temperature throughout the sampling.

Once the sampling is finished, the transfer device is moved, for use of the sampled biological material. Thus, within the framework of a medically assisted fertilisation procedure, the biological material is transferred in order to be inserted into the uterine cavity of a patient for injection. Transport of the transfer device from the temperature-controlled environment such as it exists in the cabinet, to the patient, can take between a few seconds to a few minutes. A temperature drop in the transfer device, which could reach 20° C., therefore takes place during the transport. This temperature drop is detrimental to the biological material transferred. In the specific case of embryo transfer, for several reasons it is considered that this can affect satisfactory implantation thereof in the uterus, and thus reduce the chances of success of the fertilization process.

The present invention proposes a solution to this problem, namely a protection device configured to allow the transport of the medical device while still maintaining the temperature in said medical device at a predefined temperature, for example close to body temperature.

To this end, the invention relates to a protection device for a medical device comprising a substantially cylindrical body and a tube, the body and the tube being hollow and open at their ends, the body including a thermally insulating distal part and assembly means, the tube including complementary assembly means, the body being capable of sliding on the tube in a longitudinal direction from a first position corresponding to a sampling configuration of the protection device to a second position corresponding to a protection configuration of the protection device, the assembly means of the body and the complementary assembly means of the tube being capable of immobilizing the body in longitudinal translation in the first position and the second position.

The protection device thus constituted allows the storage of a medical device up to the moment of the medical intervention, for example up to the moment of the injection or insertion of a biological material into the body of a patient. Once a medical device is inserted into the protection device, an operator can easily place the protection device in one or other of the sampling and protection configurations according to need.

In particular, the thermally insulating distal part allows storage of at least a portion of the medical device in the distal part. In fact, the protection device can be brought to a configuration allowing the distal part of the body to cover a portion of the medical device to be maintained within a predefined temperature range.

According to a characteristic, the length of the protection device in the protection configuration is greater than the length of the protection device in the sampling configuration.

By modifying the length of the protection device, and in particular by increasing the length, the protection device thus makes it possible to protect a portion of the medical device by sliding the body from the first position to the second position.

According to a characteristic, the distal part of the body is formed by a double cylindrical wall.

The thermal insulation properties are thus improved.

According to a characteristic, the protection device comprises a hollow cylinder arranged in the distal part of the body, the cylinder being produced from a material having a high thermal conductivity, in particular aluminium.

Producing the cylinder from a material having a high thermal conductivity makes it possible to heat the cylinder easily by thermal conduction. The protection device can for example be heated before use, in particular by placing the cylinder in contact with a heat source. For example, a pre-heated rod or plug can be inserted into the cylinder.

According to a characteristic, the cylinder can be produced from a material having a high thermal capacity. The cylinder thus makes it possible to store heat, which also helps to maintain the distal part of the body at a predefined temperature.

According to a characteristic, the cylinder has a length at least equal to a length of the distal part of the body.

The cylinder thus makes it possible to heat all the distal part of the body thoroughly and evenly.

According to a characteristic, in the second position, the assembly means of the body and the complementary assembly means of the tube are capable of immobilizing the body irreversibly in longitudinal translation.

Therefore an operator using the protection device can no longer position the body in the first position. The protection device is thus single-use, which makes it possible to reduce the risks of contamination.

According to a characteristic, the assembly means of the body comprise a first slot and a second slot, the complementary assembly means of the tube comprising a stud, the stud being capable of nesting in the first slot when the protection device is in the sampling configuration and nesting in the second slot when the protection device is in the protection configuration.

The body is thus immobilized in longitudinal translation when the body is in the first position or the second position.

According to a characteristic, the stud comprises a stop face abutting against the stop faces respectively of the first and second slots when the body is respectively in the first and second positions, the stop face of the first slot being inclined with respect to the stop face of the stud so as to allow sliding of the body from the first position to the second position, the stop face of the second slot being parallel to the stop face of the stud so as to immobilize the body irreversibly in the second position.

By virtue of the inclination of the stop face of the first slot, the stud can be disengaged from the first slot in order to position the body in the second position. Conversely, the stop face of the second slot parallel to the stop face of the stud prevents the stud from coming out of the second slot, which ensures immobilization of the protection device in the protection configuration. The stop face of the second slot is normal to the direction of sliding of the body, or longitudinal translation.

According to a characteristic, the assembly means of the body include means for guiding in translation and immobilizing in rotation capable of engaging with complementary means for guiding in translation and immobilizing in rotation of the tube when the body is in the first position or in the second position so as to immobilize the body in rotation about a longitudinal axis and to guide the sliding of the body on the tube.

According to a characteristic, the tube comprises fastening means capable of fastening the tube on the medical device.

The fastening means thus make it possible to immobilize any movement of the tube with respect to the medical device during handling of the protection device.

According to a characteristic, the body includes a proximal part communicating with the distal part via an interface opening, the proximal part having an internal cross section narrowing towards the interface opening.

The narrowing of the internal cross section of the proximal part towards the interface opening makes it possible to direct the medical device when it is inserted into the protection device. In particular, this facilitates the insertion of a part of the transfer device into the distal portion of the protection device.

The invention also relates to a medical transfer assembly comprising a protection device having the aforementioned characteristics and a transfer device. The transfer device comprises a catheter and a syringe mounted at a proximal end of the catheter, the transfer device being arranged partly in the tube of the protection device, the catheter comprising a hollow conduit, a distal portion of the conduit extending beyond the distal part of the body so as to be situated outside the protection device when the protection device is in the sampling configuration, the distal portion of the conduit being arranged inside the distal part of the body when the protection device is in the protection configuration.

It is thus possible to sample an element, for example biological material, when the protection device is in the sampling configuration, since the distal portion is arranged outside the protection device. In particular, said element can be sampled into the catheter by means of the syringe. More specifically, said sampled element can be kept in the distal portion of the conduit of the catheter. In this way, the protection device can be arranged in the protection configuration in which the thermally insulating distal part of the body covers the distal portion containing the sampled element.

Sampling the element can be carried out in a heated cabinet. The protection device placed in the protection configuration is removed from the heated cabinet to be transported to the site of the medical intervention. The distal portion of the medical device is thus maintained at a predefined temperature and is thus protected from the temperature variations that could be detrimental to the sampled element. The medical device is thus protected from the temperature variations throughout the duration of its handling.

According to a characteristic, the transfer device includes a secure connector, the secure connector comprising at a proximal end mounting means capable of fastening the syringe irreversibly to the secure connector.

The secure connector makes it possible to mount the syringe securely to the catheter and to prevent them from becoming detached during handling of the transfer device.

According to a characteristic, the secure connector is a screw connector comprising a distal end bore, the catheter being capable of being screwed to and unscrewed from the distal end of the secure connector.

Unlike the syringe, which is mounted irreversibly to the secure connector, the catheter can be mounted and removed from the connector by screwing and unscrewing. However, the catheter cannot be detached from the connector simply by pulling on the catheter.

According to a characteristic, the fastening means of the tube are capable of fastening the tube on the secure connector.

Fastening the tube onto the transfer device makes it possible to immobilize any movement of the tube with respect to the transfer device, while avoiding deterioration of the catheter.

Moreover, fastening the tube onto the secure connector instead of a fastening onto the syringe avoids deterioration of the syringe.

According to a characteristic, the distal portion of the conduit is capable of receiving biological material, in particular an embryo.

Finally, the invention relates to a method for the transfer of an element to be sampled by means of the medical transfer assembly having the characteristics described above, and comprising the following steps:
arranging the protection device in the sampling configuration;
arranging a distal end of the conduit of the catheter in a dish containing the element to be sampled;
sampling said element to be sampled into the transfer device by creating a pressure drop in the transfer device by means of the syringe; and
arranging the protection device in the protection configuration.

Other features and advantages of the invention will become more apparent from the following description, with reference to the attached drawings, given by way of non-limitative examples:

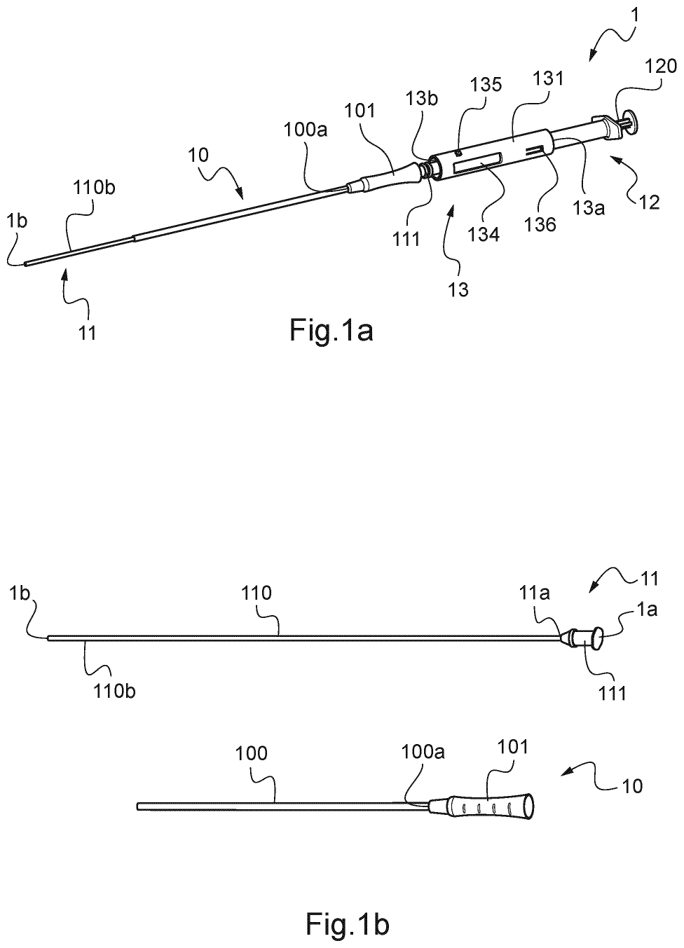

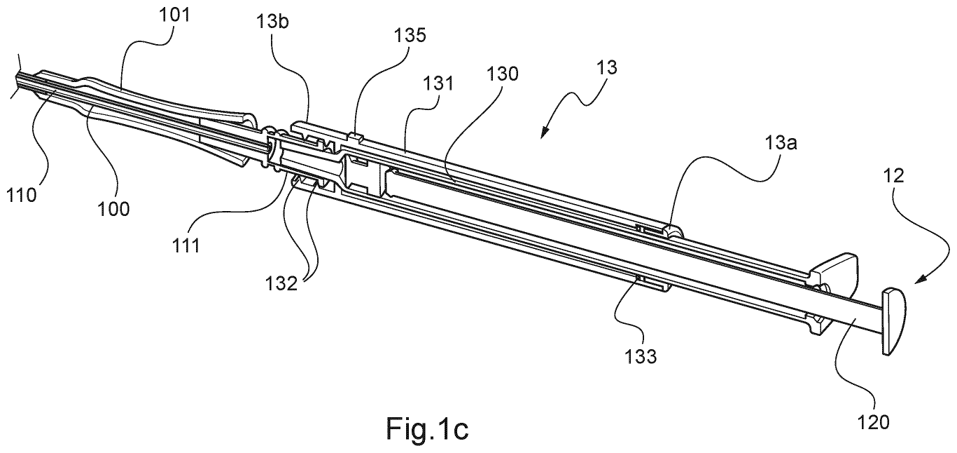

FIG. 1a is a perspective view of a transfer device capable of being mounted to a protection device according to the invention;

FIG. 1b is an exploded view of a catheter assembly in FIG. 1a;

FIG. 1c is a cross section view of the transfer device in FIG. 1a;

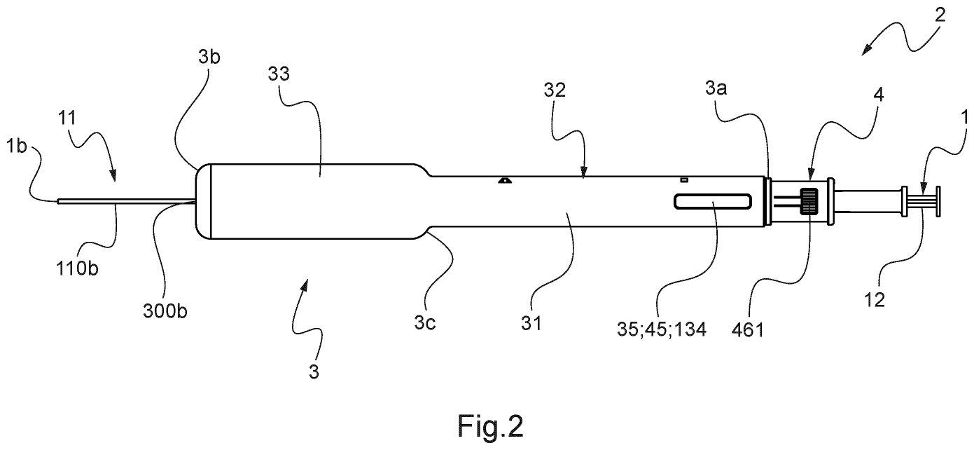

FIG. 2 is a perspective view of a protection device in an embodiment according to the invention on which is mounted the transfer device in FIG. 1a, the protection device being in the sampling configuration;

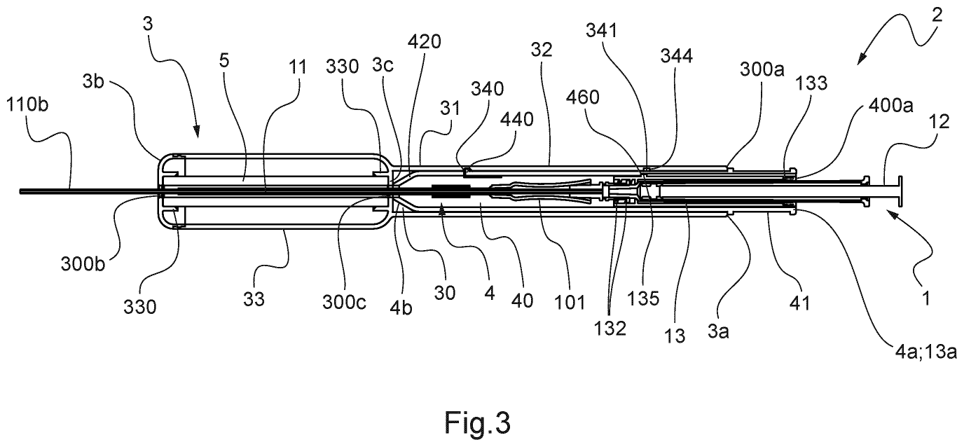

FIG. 3 is a cross section view along a first plane in FIG. 2;

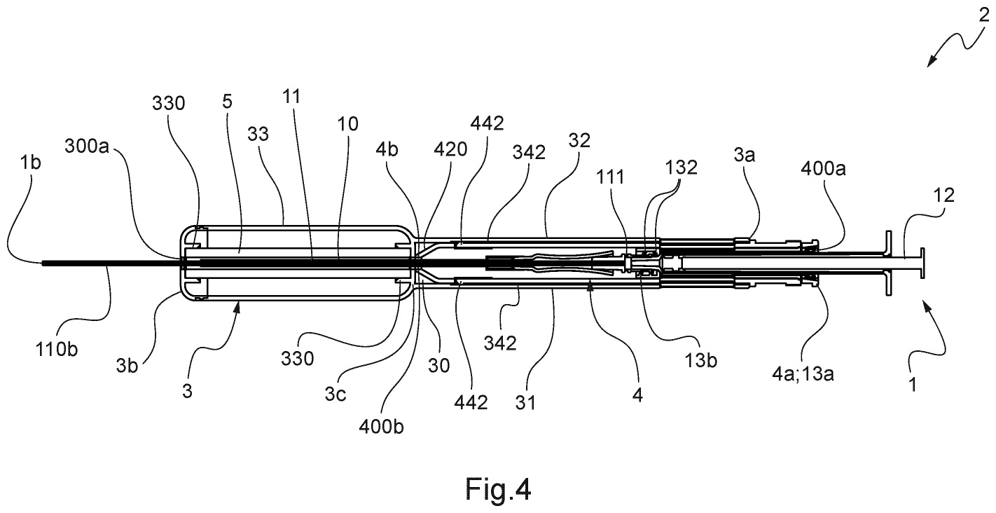

FIG. 4 is a cross section view along a second plane in FIG. 2;

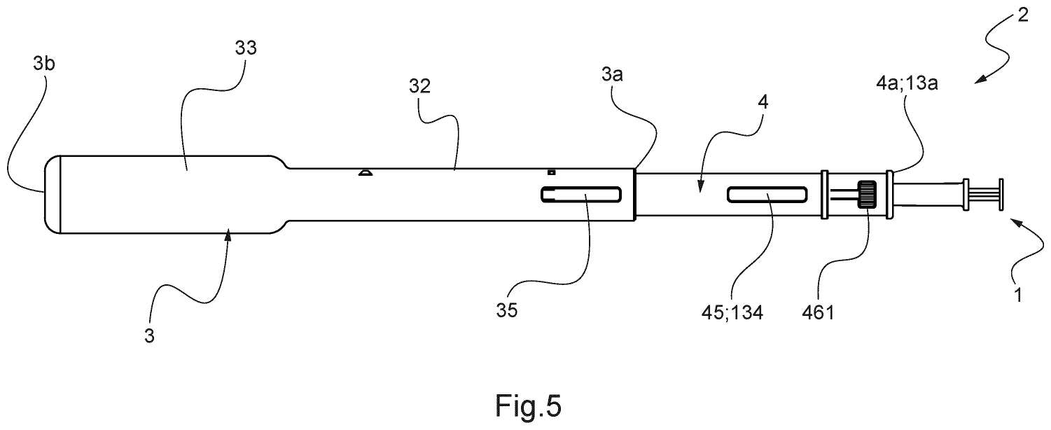

FIG. 5 is a perspective view of the protection device in FIG. 2 in the protection configuration;

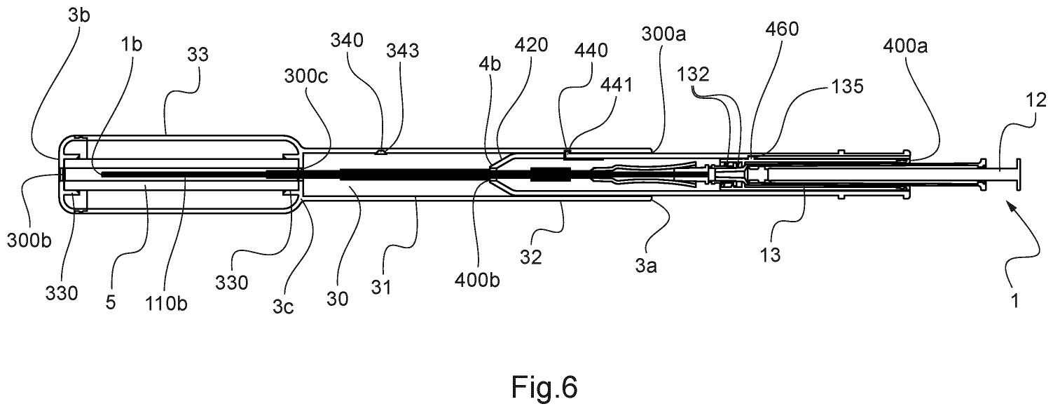

FIG. 6 is a cross section view along the first plane in FIG. 5; and

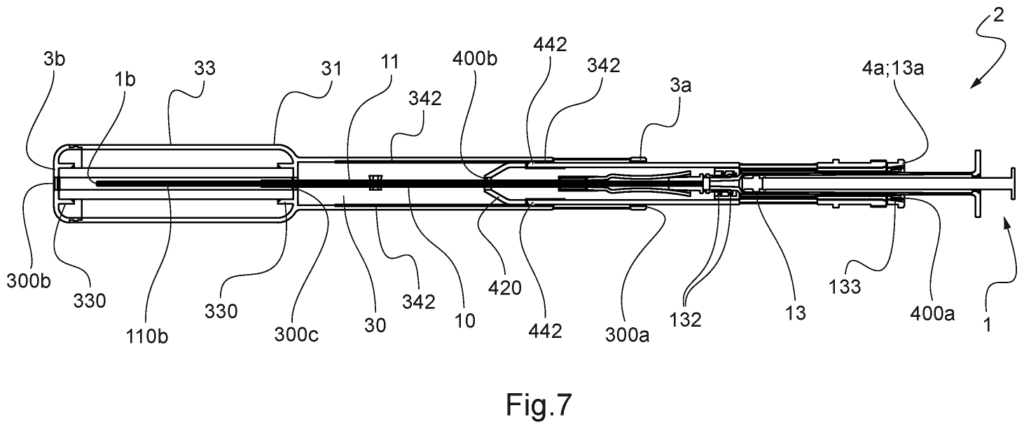

FIG. 7 is a cross section view along the second plane in FIG. 5.

In the description, the term “proximal” refers to a situation close to an operator, while the term “distal” refers to an opposite situation. In the example described hereinafter relating to a transfer of an embryo, the distal part refers to the part of the transfer device intended to be inserted into the uterine cavity of a patient.

The terms “inner surface” and “outer surface” refer to the lateral surfaces of an element, defining between them the thickness of said element. Moreover, by “inner volume” is meant the volume defined or delimited between the inner surface. Finally, the inner diameter of a piece refers to the diameter of the inner surface of said piece.

FIGS. 1a, 1b and 1c represent a medical device. Here, the medical device is a transfer device 1 of biological material, in particular of an embryo, but of course may be any other type of medical device. FIG. 1c is a cross section view along a median plane of FIG. 1a.

The transfer device 1 includes on the one hand a catheter or a catheter assembly, and on the other hand a syringe 12, both known per se. The transfer device 1 has the feature of also including a secure connector 13.

In this example, the transfer device 1 includes a catheter assembly constituted by a first catheter 10 and a second catheter 11 intended to engage with one another. The first catheter 10 and the second catheter 11 are open at their ends.

The first catheter 10 comprises a first hollow conduit 100 and an insertion sleeve 101.

The first conduit 100 is here produced in a non-transparent material. The first conduit 100 is here produced in a flexible material.

A proximal end 100a of the first hollow conduit 100 opens into the sleeve 101. The sleeve 101 has a substantially cylindrical shape. The sleeve 101 is intended to remain outside the body cavity of the patient.

The second catheter 11 includes a second, conduit 110 that is transparent and hollow, and a handle 111.

The second conduit 110 has a diameter that is smaller than the diameter of the first conduit 100 of the first catheter 10. This allows the first conduit 100 to accommodate the second conduit 110. The second conduit 110 is also produced from a flexible material. The second conduit 110 is produced from a transparent material.

The second conduit 110 is intended to contain biological material, for example an embryo, intended to be inserted into the neck of the uterus of the patient.

The handle 111 extends a proximal end 11a of the second conduit 110.

The length of the second catheter 11 is greater than the length of the first catheter 10, so that when the second catheter 11 is inserted into the first catheter 10, the handle 111 and a distal portion 110b of the second conduit 110 extends beyond the first catheter 10.

The catheter assembly is intended to be inserted into the uterine cavity of the patient to make it possible to reach the neck of the uterus with a distal end 1b of the catheter assembly and in particular of the second conduit 110. Insertion of the catheter assembly into the neck of the uterus is facilitated by the flexibility of the first and second conduits 100, 110. The transparency of the second conduit 110 allows an operator to view the biological material during sampling.

In a different example of the prior art, also known, the transfer device 1 includes a single catheter.

The secure connector 13 comprises a proximal end 13a to which the syringe 12 is mounted and a distal end 13b to which the catheter is mounted, in particular here, the second catheter 11. In other words, the second catheter 11 and the syringe 12 are assembled with one another by means of the secure connector 13.

The secure connector 13 is here a hollow cylinder. The secure connector 13 comprises a bore formed at the distal end 13b. In particular, the secure connector 13 is a screw connector or threaded sleeve connector. The bore comprises rings 132 formed on the inside of the secure connector 13. The rings 132 project from an inner wall 130 of the secure connector 13.

Furthermore, the secure connector 13 includes mounting means 133 at its proximal end 13a. The mounting means 133 are formed in this example by a resilient annular element or annular clip projecting from the inner wall 130 of the secure connector 13.

The secure connector 13 comprises an aperture, called first aperture 134, capable of exposing a part of the syringe 12 when the transfer device 1 is assembled.

The connector also comprises three fingers, called first finger 135, second finger 136 and third finger (not shown in the Figures), projecting from an outer wall 131 of the secure connector 13. The second finger 136 and the third finger are formed symmetrically with respect to the first finger 135.

The syringe 12 is a conventional syringe similar to those commonly used in medical laboratories for the sampling and transfer of elements. The syringe 12 makes it possible to carry out a sampling, an injection or an insemination of an element, for example biological material, by creating a pressure drop or increase in the catheter assembly.

During assembly of the transfer device 1, the syringe 12 is mounted at the proximal end 13a of the secure connector 13. In particular, a part of the syringe 12 is fastened to the secure connector 13 by virtue of the mounting means 133. In the example described, the resilient element constituting the mounting means 133 deforms by elasticity during the insertion of the syringe 12 into the secure connector 13.

Fastening the syringe 12 onto the secure connector 13 is irreversible, that is to say that the mounting means 133 do not allow the syringe 12 to be removed once the latter has been fastened to the secure connector 13.

The second catheter 11 is then mounted to the secure connector 13. Here, the second catheter 11 is screwed to the distal end 13b of the secure connector 13. The handle 111 is in particular screwed onto the rings 132. The second catheter 11 can be unscrewed from the secure connector 13. Thus, unlike the fastening of the syringe 12 onto the secure connector, fastening the second catheter 11 to the secure connector is reversible. However, the second catheter 11 cannot be removed from the secure connector 13 by pulling on it only.

The bore, and in particular the rings 132 as well as the mounting means 133 formed in the secure connector 13 make it possible to avoid the second catheter 11 becoming detached from the syringe 12.

FIGS. 2 to 7 represent a protection device 2 for a medical device in an embodiment according to the invention.

Le protection device 2 comprises a body 3 and a tube 4. The body 3 and the tube 4 are hollow and open at their ends. The body 3 is capable of sliding on the tube 4 in a longitudinal direction between a first position and a second position. The first position and the second position are end of travel positions, here also called end positions of longitudinal translation. The body 3 comprises a proximal opening 300a formed at a proximal end 3a and suitable for the insertion of the tube 4. The body 3 comprises a distal opening 300b formed at a distal end 3b opposite to the proximal end 3a.

The body 3 comprises an outer surface 31 and an inner surface 30. The inner surface 30 of the body 3 delimits an inner space capable of receiving the tube 4.

The body 3 has a substantially cylindrical shape. In the description, the longitudinal direction refers to a direction parallel to a longitudinal axis or axis of revolution about which the body 3 extends.

The body 3 comprises a proximal part 32 and a distal part 33. The proximal part 32 and the distal part 33 communicate with one another at one end of the interface 3c via an interface opening 300c.

The interface opening 300c has a diameter less than the inner diameter of the proximal part 32. In particular, the interface opening 300c is smaller than the proximal opening 300a of the body 3. In the embodiment represented, the portion of the body 3 adjacent to the distal part 33 has an internal cross section narrowing towards the interface opening 300c.

Furthermore, the interface opening 300c has a diameter at least equal to the diameter of the second catheter 11 of the transfer device 1, and in particular to the diameter of the distal portion 110b, so as to allow the passage of the second conduit 110.

The distal part 33 is thermally insulating. In the example represented, the distal part 33 is formed by a double wall, which confers on it thermal insulation properties.

The distal part 33 comprises receiving elements 330. The receiving elements 330 are here formed by a first hollow cylindrical projection extending from the interface end 3c to the distal end 3a, and by a second hollow cylindrical projection from the distal end 3a to the interface end 3c.

The body 3 comprises assembly means 340, 341, 342.

The assembly means 340, 341, 342 of the body 3 comprise means capable of immobilizing the longitudinal translation of the body. Here, the assembly means of the body comprise a first slot 340 and a second slot 341. The first slot 340 and the second slot 341 are arranged in an inner surface 30 of the body 3. The first slot 340 comprises a stop face 343 and the second slot 341 comprises a stop face 344. The distance separating the first slot 340 from the second slot 341 is equal to the travel distance of the body 3 or distance covered by the body 3 when it slides from the first position to the second position.

The assembly means of the body 3 also comprise means for guiding in translation and immobilizing in rotation about the longitudinal direction. In this example, the assembly means comprise a groove 342 formed in the inner surface 30 of the body 3.

The body 3 comprises at least one aperture, called second aperture 35. The second aperture 35 is capable of exposing a part of the body 3 and/or of the tube 4. The second aperture 35 has a substantially rectangular shape.

The tube 4 is capable of being arranged in the proximal part 32 of the body 3. In other words, here, the tube 4 does not extend into the distal part 33 regardless of the position in which the body 3 is situated.

The tube 4 comprises an inner surface 40 and an outer surface 41. The inner surface 40 of the tube 4 delimits an inner space or volume capable of receiving the medical device.

The tube 4 comprises a proximal end 4a and a distal end 4b. The tube 4 comprises a distal opening 400b formed at the distal end 4b of the tube 4 and opposite to a proximal opening 400a formed at the proximal end 4a of the tube 4. The proximal opening 400a of the tube 4 is suitable for the insertion of the medical device. The distal opening 400b of the tube 4 is narrow with respect to the proximal opening 400a of the tube 4. A distal portion of the tube 4 has a cross section narrowing towards the distal opening 400b. Here, the distal portion of the tube 4 has a substantially frustoconical shape, the distal opening 400b forming the smallest base of the truncated cone. The distal opening 400b of the tube 4 has a diameter at least equal to the diameter of the second catheter 11 of the transfer device 1, in particular to the diameter of the distal portion 110b.

The tube 4 comprises a chamfered front face 420 extending to the distal end 4b of the tube 4. The chamfered shape of the front face 420 facilitates the insertion of the tube 4 into the body 3 during the mounting of the protection device.

The tube 4 comprises complementary assembly means 440, 442. The complementary assembly means of the tube 4 are capable of engaging with the assembly means of the body 3 so as to immobilize the longitudinal translation of the body 3 on the tube 4 as well as the rotation of the body 3 about the longitudinal direction. In the embodiment described, by engaging with one another, the assembly means of the body and the assembly means of the body also make it possible to guide the sliding of the body 3 on the tube 4.

The complementary assembly means comprise a stud 440 formed in the outer surface 41 of the tube 4. The stud 440 is shaped so as to allow nesting via complementarity of shapes in the first slot 340 and the second slot 341 of the body 3. The stud 440 comprises a stop face 441 capable of abutting against the stop face 343 of the first slot 340 when the body 3 is in the first position, and abutting against the stop face 344 of the second slot 341 when the body 3 is in the second position. The stop face 344 of the first slot 340 is inclined with respect to the stop face 441 of the stud 440. The stop face 344 of the second slot 341 is parallel to the stop face 441 of the stud 440.

The complementary assembly means of the tube 4 also comprise two projections 442. The projections 442 are shaped symmetrically with respect to the stud 440. By engaging with the groove 342 of the body 3, the projections 442 make it possible to guide the sliding of the body 3 on the tube 4 as well as the immobilization in rotation of the body 3 about the longitudinal direction. The projections 442 of the tube 4 are spaced apart by a distance less than the length of the groove 342 of the body 3.

Moreover, the engagement between the groove 342 of the body 3 and the projections 442 of the tube 4 make it possible to place the second aperture 35 of the body 3 facing the third aperture 45 of the tube 4 when the body 3 is in the first position.

The tube 4 also comprises an aperture, called third aperture 45. The third aperture 45 of the tube 4 has the same shape and the same dimensions as the second aperture 35 of the body 3.

The body 3 and the tube 4 can be produced from plastic material, for example by injection or moulding. This makes it possible to easily confer on them the desired shape and dimensions. The use of plastic allows a light protection device to be obtained.

The tube 4 comprises fastening means capable of fastening the tube 4 on the medical device. The fastening means are capable of preventing rotation of the tube 4 about the longitudinal direction as well as sliding in the longitudinal direction of the tube 4 on the transfer device 1. In this example, the tube 4 comprises a hollow 460 opening onto the inner surface 40 and two buttons 461 formed symmetrically with respect to the hollow 460.

The protection device 2 comprises a hollow cylinder 5 arranged in the distal part 33 of the body 3. The cylinder 5 and the body 3 are thus firmly fixed to one another. The cylinder 5 is housed between the receiving elements 330 of the body 3. The cylinder 5 is produced from a material having a high thermal conductivity. Preferably, the cylinder 5 is produced with a material having a thermal conductivity at least equal to 150 W·m⁻¹·K⁻¹. The cylinder can be produced from aluminium or else from silver or gold.

In another embodiment, the cylinder 5 has a reinforcement capable of accommodating a stud formed in the distal part 33 of the body 3.

The cylinder 5 is open at its ends and comprises a passage capable of accommodating for example a heating plug (not shown). The plug can have the form of a pre-heated cylindrical rod capable of being inserted into the cylinder 5 before use of the protection device. This makes it possible to heat the cylinder 5 and thus to heat the distal part 33 of the body 3.

In another embodiment, at least a part of the cylinder 5 extends beyond the distal part 33 of the body, outside the body 3. The cylinder 5 then has a length greater than a length of the distal part 33 of the body 3 so that a part of the cylinder 5 extends outside the body 3. The cylinder 5 can thus be heated by conduction by placing it in contact with a heat source such as a hot-plate.

The passage formed in the cylinder 5 also makes it possible to accommodate a part of the transfer device 1. In particular, the passage has a cylindrical shape and has a diameter at least equal to the diameter of the second catheter 11, in particular to the diameter of the distal portion 110b.

The protection device 2 is configured to adopt a sampling configuration and a protection configuration.

FIGS. 2, 3, 4 represent the protection device 2 in sampling configuration in which the assembly means 340, 341, 342 of the body 3 engage with the complementary assembly means of the tube 4 so as to place the body 3 in the first position. FIG. 2 is a perspective view of the protection device in the sampling configuration. FIG. 3 is a cross section view along a first median plane of the protection device in FIG. 2, the first plane passing through the longitudinal axis. FIG. 4 is a cross section view along a second median plane of the protection device in FIG. 2, the second plane passing through the longitudinal axis and substantially orthogonal to the first plane. In the embodiment example described, the projections 442 of the tube 4 engage with the groove 342 of the body 3 so as to position the second aperture 35 of the body 3 facing the third aperture 45 of the tube 4. The stud 440 of the tube 4 is nested in the first slot of the body 3. The stop face 441 of the stud 440 is thus in abutment against the stop face 343 of the first slot 340. The distal end 4b of the tube 4 and the interface end 3c extend substantially in one and the same transverse plane.

FIGS. 5, 6, 7 represent the protection device 2 in protection configuration in which the assembly means 340, 341, 342 of the body 3 engage with the complementary assembly means of the tube 4 so as to place the body 3 in second position. FIG. 5 is a perspective view of the protection device in the protection configuration. FIG. 6 is a cross section view along the first median plane of the protection device in FIG. 5. FIG. 7 is a cross section view along the second median plane of the protection device in FIG. 5. In the embodiment described, the projections 442 engage with the groove 342 and the stud 440 of the tube 4 is nested in the second slot 341 of the body 3. The stop face 441 of the stud 440 is thus in abutment against the stop face 344 of the second slot 341.

The protection device 2 passes from the sampling configuration to the protection configuration by sliding of the body 3 on the tube 4. In other words, an operator or practitioner pulls on the body 3 so as to make the body 3 pass from the first position to the second position. In the example described, the operator pulls on the body 3 so as to disengage the stud 440 from the first slot 340 of the tube 4. As the stop face 343 of the first slot 340 is inclined with respect to the stop face 441 of the stud 440, the stud can indeed be disengaged from the first slot 340 by applying a traction force to the body 3.

The body 3 is then no longer immobilized in longitudinal translation and can thus slide on the tube 4 up to the second longitudinal translation end position. The projections 442 of the tube 4 engaging with the groove of the body 3 make it possible to guide the sliding of the body 3 on the tube 4. The stud 440 of the tube 4 is accommodated in the second slot 341 of the body 3. As the stop face 344 of the second slot 341 is parallel to the stop face 441 of the stud 440, the stud 440 cannot be disengaged from the second slot 341. The body 3 is thus immobilized in the second position and as a result the protection device is immobilized in the protection configuration.

Sampling an element takes place according to the following steps.

The element to be sampled, contained in a Petri dish, is arranged for example in a semi-closed heated cabinet. The cabinet is heated for example by means of a horizontal hot-plate. The heated cabinet comprises two through-holes suitable for allowing the arms of the operator to pass through, so as to permit handling of the medical material. The heating plug can be pre-heated, for example in an oven or by being arranged on the hot-plate of the cabinet. Once heated, the heating plug can then be arranged in the cylinder 5 so as to heat the distal part 33 of the body 3.

In the alternative embodiment (not shown) where a part of the cylinder projects from the distal part of the body, the protection device 2 alone, not mounted on the transfer device 1, can be arranged in the cabinet beforehand, so that the cylinder 5 of the protection device 2 is in contact with the hot-plate. To this end, the protection device 2 can be arranged vertically on the hot-plate. The protection device can also be heated in an oven. As the cylinder 5 of the protection device 2 has a high thermal conductivity, it then heats by conduction.

As the distal part 33 of the body 3 is thermally insulating, this allows heat to be stored in the distal part 33 of the protection device 2. The protection device 2 can be kept in the cabinet while waiting to be used.

Of course, sampling the element can be carried out in an environment other than that of the heated cabinet.

Before sampling an element, the transfer device 1 is assembled. In particular, the syringe 12 is assembled to the secure connector 13 by virtue of the mounting means 133. In the example described, the resilient element of the secure connector 13 allows irreversible fastening of the syringe 12 onto the secure connector 13. In other words, once the syringe 12 is assembled with the secure connector 13, the syringe 12 and the secure connector 13 can no longer be detached. The second catheter 11 is then assembled with the secure connector 13. The handle 111 is in particular screwed onto the rings 132. Assembly of the second catheter 11 with the syringe 12 is thus secured by virtue of the secure connector 13.

The protection device 2 is then mounted on the transfer device 1. FIGS. 2 to 7 represent the protection device 2 on which the transfer device 1 is mounted. The transfer device 1 is inserted into the protection device 2. In the embodiment example described, the narrowed cross section of the portion of the body 3 adjacent to the distal part 33 and the distal portion of the tube 4 facilitates the insertion of the second conduit 110 of the transfer device 1 into the protection device 2. In effect this allows the catheter to be guided during its insertion into the protection device 2. Of course, the transfer device 1 is not completely inserted. At least a proximal part of a piston 120 of the syringe 12 extends beyond a proximal end of the protection device 2 so as to allow sampling and injection of the element, for example biological material.

The protection device is then fastened to the transfer device. The first finger 135 of the secure connector 13 is positioned in the hollow 460 of the tube 4 so as on the one hand, to immobilize the tube 4 in rotation about the longitudinal direction and on the other hand, to position the first aperture 134 of the secure connector 13 facing the third aperture 45 of the tube 4 and the buttons 461 of the tube 4 facing the second finger 136 and third finger of the secure connector 13. By applying pressure on the buttons 461 of the tube 4, the buttons 461 are pressed in and clipped on the second finger 136 and the third finger of the secure connector 13. The tube 4 is thus immobilized in longitudinal translation.

The protection device 2 is dimensioned so that when the protection device 2 is in the sampling configuration, the distal portion 110b of the second catheter 11 extends beyond a distal end of the protection device 2. In other words, the distal portion 110b of the transfer device 1 extends outside the protection device 2. It is thus possible to sample the element to be sampled into the catheter, in particular into the second catheter 11.

In practice, the medical assembly including the protection device 2 and the transfer device 1 is transferred up to a container of the element to be sampled, for example the Petri dish. The protection device 2 is arranged in the sampling configuration. The distal end 1b of the second catheter 11 is arranged in the container. The piston 120 of the syringe 12 is drawn, so as to create a pressure drop in the second catheter 11. The biological material is thus sampled into the distal portion 110b of the second catheter 11.

As the second aperture 35 of the body 3 is arranged facing the third aperture 45 of the tube 4 and the first aperture 134 of the secure connector 13 when the protection device 2 is in the sampling configuration, the operator can thus view a part of the syringe 12, for example graduations. The operator can thus sample precisely the quantity of element desired.

Once the sampling is finished, the protection device 2 is brought to the protection configuration. The body 3 slides on the tube 4 until the distal part 33 of the body 3 completely covers the distal portion 110b of the second catheter 11. In other words, the distal portion 110b of the catheter is situated completely inside the protection device 2, in particular inside the distal part 33 of the body 3. Preferably, the sliding travel or distance of the body 3 is at least equal to the length of the distal portion 110b of the transfer device 1. By “travel” of the body 3 is meant the distance, in the longitudinal direction, covered by the body 3 from the first to the second longitudinal translation end position.

The medical assembly including the protection device 2 and the transfer device 1 can be extracted from the cabinet to be conveyed to the body of the patient. During the transport, the protection device 2 is maintained in the protection configuration. The distal portion 110b of the transfer device 1 is thus arranged completely in the distal part 33 of the protection device 2. As the distal part 33 of the protection device 2 is thermally insulating, the distal portion 110b of the transfer device 1 including the sampled element is then protected from temperature variations.

In the particular case of transfer of biological material such as an embryo, the transfer device 1 is removed from the protection device 2 only at the moment of insertion into the body of the patient. To this end, the operator unlocks the tube 4 from the transfer device 1 by unlocking the fastening means. In particular, pressure is applied on the buttons 461 of the tube 4, thus pressing on the second finger 136 and the third finger. The second finger 136 and the third finger then press the buttons 441 of the tube 4 by reaction. The transfer device can then be removed. The operator can in particular pull on the secure connector 13 so as to disengage the first finger 135 of the secure connector 13 from the hollow 460 of the tube 4.

Of course, the present invention is not limited to the embodiments described and illustrated.

The protection device has been described for a use with a transfer device as a medical device. In particular, it has been given as an example of a transfer device for biological material, such as an embryo. The protection device can be used for any other medical device, provided that maintaining a part of the medical device at a given or predefined temperature is concerned.

By way of example, the protection device can be used to protect a recoverable intra-uterine device capable of containing embryos or gametes such as that described in patent application EP3367937 filed in the name of the applicant.

The protection device can be used to protect an element other than biological material.

In addition, the protection device 2 can be used with a transfer device not including a secure connector 13.

Moreover, the transfer device as described can be used independently of the protection device, for example in association with other devices.

The assembly means of the body and the complementary assembly means of the tube can be different. By way of example, the nesting direction of the assembly means and the complementary assembly means can be reversed. In other words, in another embodiment the assembly means of the body can comprise studs and projections, and the complementary assembly means of the tube can comprise a groove and slots.

The assembly means of the body and the complementary assembly means of the tube can also engage other than by nesting.

In the embodiment described, the groove of the body 3 and the projections of the tube 4 make it possible by engaging with one another on the one hand to guide the sliding of the body 3 on the tube 4 and on the other hand to immobilize the rotation of the body 3 about the longitudinal direction. However, the means for immobilizing the body in rotation about the longitudinal direction and the means for guiding the sliding of the body on the tube can be dissociated. In the case in point, the body and the tube can comprise on the one hand first means engaging with one another to allow the body to be immobilized in rotation, and on the other hand second means engaging with one another to allow the sliding of the body on the tube to be guided.

The proximal part 32 of the body 3 can thus for example comprise means for immobilization in rotation about the longitudinal direction, and at least one guide element capable of facilitating the sliding of the body 3 on the tube 4. The guide element forms a projection inside the body. In other words, the cross section of the body 3 is narrowed at the level of the guide element. The guide element is in abutment against the tube and can have an annular shape.

The fastening means of the tube 4 on the medical device can comprise a locking screw. The locking screw extends in a radial or transverse direction. The locking screw comprises a head and a shank. The locking screw can be configured to adopt a locking position in which the locking screw passes through the tube 4 and an unlocking position in which the locking screw frees the inner space defined by the inner surface 40 of the tube 4. In particular, the shank is capable of passing through the tube 4 when the locking screw is in locking position and conversely, to free the inner space of the tube 4 when the locking screw is in the unlocking position.

The fastening means of the tube can include resilient clips projecting from the inner surface of the tube, capable of holding the tube fastened on the medical device. The clips can be of the type of those conventionally used in vehicle cigarette lighters.

The protection device can be combined with Internet of Things technology for example by using sensors connected to an external system such as a computer. The sensors can then send to the computer items of information to be utilized or processed, such as the temperature in the protection device or an alarm signalling a temperature drop below a predefined threshold.

The present invention proposes a protection device having an optimized structure allowing the protection of at least a part of a medical device and thus avoiding temperature variations within said part of the medical device.

1. A protection device for medical device comprising a substantially cylindrical body and a tube, said body and tube being hollow and open at their ends, wherein
the body comprises a thermally insulating distal part and assembly means,
the tube comprises complementary assembly means, and further wherein
the body is capable of sliding on the tube in a longitudinal direction from a first position corresponding to a sampling configuration of the protection device to a second position corresponding to a protection configuration of the protection device, and the assembly means of the body and the complementary assembly means of the tube are capable of immobilizing the body in longitudinal translation in the first position and the second position.

2. The protection device according to claim 1, wherein the length of the protection device in the protection configuration is greater than the length of the protection device in the sampling configuration.

3. The protection device according to claim 1, wherein the distal part of the body is formed by a double cylindrical wall.

4. The protection device according to claim 1, wherein the protection device comprises a hollow cylinder arranged in the distal part of the body, said cylinder being produced from a material having a high thermal conductivity.

5. The protection device according to claim 1, wherein the assembly means of the body and the complementary assembly means of the tube are capable of immobilizing the body irreversibly in longitudinal translation in the second position.

6. The protection device according to claim 1, wherein the assembly means of the body comprise a first slot and a second slot, the complementary assembly means of the tube comprise a stud, said stud being capable of nesting in the first slot when the protection device is in the sampling configuration and nesting in the second slot when the protection device is in the protection configuration.

7. The protection device according to claim 6, wherein said stud comprises a stop face abutting against stop faces respectively of the first and second slots when the body is respectively in the first and second positions, the stop face of the first slot being inclined with respect to the stop face of the stud so as to allow sliding of the body from the first position to the second position, the stop face of the second slot being parallel to the stop face of the stud so as to immobilize the body irreversibly in the second position.

8. The protection device according to claim 1, wherein the assembly means of the body comprise means for guiding in translation and immobilizing in rotation capable of engaging with complementary means for guiding in translation and immobilizing in rotation of the tube when said body is in the first position or in the second position, so as to immobilize the body in rotation about a longitudinal axis and to guide the sliding of the body on the tube.

9. The protection device according to claim 1, wherein the tube comprises fastening means capable of fastening the tube on the medical device.

10. The protection device according to claim 1, wherein the body comprises a proximal part communicating with the distal part via an interface opening said proximal part having an internal cross section narrowing towards said interface opening.

11. A medical transfer assembly comprising a protection device according to claim 1 and a transfer device, said transfer device comprising a catheter and a syringe mounted at a proximal end of the catheter, said transfer device being arranged partly in the tube of the protection device, said catheter comprising a hollow conduit, a distal portion of the conduit extending beyond the distal part of the body so as to be situated outside the protection device when the protection device is in the sampling configuration, said distal portion of the conduit being arranged inside the distal part of the body when the protection device is in the protection configuration.

12. The medical transfer assembly according to claim 11, wherein the transfer device further comprises a secure connector, the secure connector comprising at a proximal end assembly means capable of fastening the syringe irreversibly to the secure connector.

13. The medical transfer assembly according to claim 12, wherein the secure connector is a screw connector comprising a distal end bore, said catheter being capable of being screwed to and unscrewed from said distal end of the secure connector.

14. The medical transfer assembly according to claim 12, wherein the tube of the protection device comprises fastening means capable of fastening the tube on the medical device, wherein the fastening means of the tube are capable of fastening the tube on the secure connector.

15. The medical transfer assembly according to claim 11, wherein the distal portion of the conduit is capable of receiving biological material.

16. A method for the transfer of an element to be sampled by means of the medical transfer assembly according to claim 11, comprising the following steps:
arranging the protection device in the sampling configuration;
arranging a distal end of the conduit of the catheter in a dish containing the element to be sampled;
sampling said element to be sampled into the transfer device by creating a pressure drop in the transfer device by means of the syringe; and
arranging the protection device in the protection configuration.

17. The protection device according to claim 4, wherein the material having a high thermal conductivity is aluminium.

18. The medical transfer assembly according to claim 15, wherein the distal portion of the conduit is capable of receiving an embryo.
